(19) United States
(12) Patent Application Publication
(10) Pub. No.: US 2021/0275467 A1
(43) Pub. Date: Sep. 9, 2021

(54) TUMOR REDUCTION FORMULATIONS AND METHODS OF USE THEREOF

(71) Applicant: TYME, INC, Bedminster, NJ (US)

(72) Inventors: Steven Hoffman, Mahwah, NJ (US); John Rothman, Lebanon, NJ (US)

(73) Assignee: TYME, INC, Bedminster, NJ (US)

(21) Appl. No.: 17/259,477

(22) PCT Filed: Jul. 2, 2019
(86) PCT No.: PCT/US2019/040264

§ 371 Date: Jan. 11, 2021

Related U.S. Application Data
(60) Provisional application No. 62/695,614, filed on Jul. 9, 2018.

(51) Int. Cl. A61K 31/08; A61K 31/4015; A61K 31/045; A61K 31/575

(52) U.S. Cl. CPC A61K 31/08; A61K 31/4015; A61K 31/167; A61K 31/575; A61K 31/045

(57) ABSTRACT

The invention relates to tumor reducing compositions and methods thereof. Specifically, the invention relates to compositions comprising a combination of a sclerosing agent and a penetrating agent.

### CROSS-REFERENCE TO RELATED APPLICATIONS

This application claims priority to and the benefit of U.S. Provisional Patent Application 62/695,614, filed Jul. 9, 2018, which is incorporated by reference herein in its entirety.

### FIELD OF THE INVENTION

The invention relates to tumor reducing compositions and methods of use thereof. Specifically, the invention relates to compositions comprising a combination of a sclerosing agent and a penetrating agent.

### BACKGROUND OF THE INVENTION

Cancer remains among the leading causes of death throughout the world. According to statistics provided by the National Cancer Institute, the number of new cancer cases per year is expected to rise to 23.6 million by 2030.

Cancer can start almost anywhere in the body, but in all types, some of the body's cells begin to divide without ceasing and spread into surrounding tissues. Normally, cells grow and divide to form new cells as the body needs them and when the cells grow old or become damaged, they die, with new cells taking their place. When cancer develops, however, this orderly process breaks down, so as cells become more and more abnormal, old or damaged cells survive when they should die, and new cells form when they are not needed. These extra cells can divide without stopping, thereby forming tumors. Many cancers form solid tumors, abnormal masses of tissue that result from this uncontrolled cell division or lack of orderly cell death.

Cancerous tumors are malignant, meaning they can spread into, or invade, nearby tissues. In addition, as these tumors grow, some cancer cells can break off and travel to distant places in the body through the blood or the lymph system and form new tumors far from the original tumor.

Tumors may also be benign (not cancerous) and although benign tumors do not spread into or invade surrounding tissues, some can be quite large and thus can also pose serious health problems and risks. Indeed, benign brain tumors can be life threatening.

As tumors increase in size, particularly malignant tumors, vital organs can become nonfunctional. Cancer treatments today can include surgery, hormone therapy, radiation, chemotherapy, immunotherapy, and targeted therapy. Although progress continues to be made in the treatment of both benign and malignant tumors, each of these treatments has limitations and drawbacks, either in effectiveness, safety, or a combination. Many of these treatments are imprecise and at times, cancers can become resistant to them. Chemotherapeutic methods and radiation therapy can damage healthy tissue and cause numerous additional adverse effects, and surgical excision can be dangerous and ineffective when tumors grow large and become too invasive.

Accordingly, there remains a great need in the art for new and innovative methods of targeting tumors, both benign and malignant, and effectively reducing their size, slowing or halting their growth, or destroying or otherwise eliminating them completely.

### SUMMARY OF THE INVENTION

In one aspect, provided herein is a pharmaceutical composition comprising a combination of a sclerosing agent and a penetrating agent. In a related aspect, the composition is a tumor reducing composition. In another aspect, the sclerosing agent is nonaethylene glycol monododecyl ether.

In another aspect, the penetrating agent is anhydrous 1-methyl-2-pyrrolidinone. In another aspect, the sclerosing agent and the penetrating agent are present in combination in an amount effective to reduce a size of a tumor in a subject. In an aspect, the subject is a mammal. In another aspect, the mammal is a human.

In an aspect, the composition further comprises an alcohol. In another aspect, the alcohol is benzyl alcohol. In another aspect, the composition further comprises an acid. In another aspect, the acid is a bile acid. In another aspect, the bile acid is sodium deoxycholate.

In an aspect, the composition further comprises a pain reducing agent. In another aspect, the pain reducing agent is lidocaine.

In an aspect, the combination of sclerosing agent and penetrating agent present in the composition penetrates a tissue in presence of an acid.

In another aspect, provided herein is a pharmaceutical composition comprising benzyl alcohol, sodium deoxycholate, nonaethylene glycol monododecyl ether, and anhydrous 1-methyl-2-pyrrolidinone.

In another aspect, provided herein are methods of reducing tumor size in a subject by administering the compositions described herein to the subject.

In another aspect, provided herein is a method of reducing a size of a tumor in a subject, comprising contacting a tumor with a composition comprising a therapeutically effective amount of a combination of a sclerosing agent and a penetrating agent. In another aspect, provided herein is a method of reducing a size of a tumor in a subject, comprising contacting a tumor with a composition comprising a therapeutically effective amount of a combination of a sclerosing agent and a penetrating agent, and a bile acid. In an aspect, the contacting comprises intratumoral injection. In an aspect, the subject is a mammal. In another aspect, the mammal is a human. In another aspect, the tumor is a cancerous tumor.

In an aspect, the tumor is present in a tissue of the breast, prostate, lung, colon, stomach, pancreas, ovary, brain, skin, bone, fat, lymph, gastrointestinal tract, liver, or soft tissue.

In an aspect, the composition further comprises an alcohol. In another aspect, the alcohol is benzyl alcohol. In another aspect, the composition further comprises an acid. In another aspect, the acid is a bile acid. In another aspect, the bile acid is sodium deoxycholate.

In an aspect, the composition further comprises a pain reducing agent. In another aspect, the pain reducing agent is lidocaine. In another aspect, the combination of sclerosing agent and penetrating agent present in the composition penetrates a tissue in presence of an acid.

In another aspect, the composition comprises benzyl alcohol, sodium deoxycholate, nonaethylene glycol monododecyl ether, and anhydrous 1-methyl-2-pyrrolidinone.

In an aspect, tumor size of the tumor contacted with the composition is reduced by about 25% to about 100% compared to a reduction of tumor size of a tumor contacted with a control. In another aspect, the tumor contacted with the composition comprises an increase in intratumoral necrosis compared to a tumor contacted with a control. In another aspect, the control comprises bacteriostatic water.

In another aspect, provided herein is a method of treating a lesion in a subject, comprising contacting the lesion with any of the pharmaceutical compositions as described herein. In an aspect, the lesion is present in a tissue of the breast, prostate, lung, colon, stomach, pancreas, ovary, brain, skin, bone, fat, lymph, gastrointestinal tract, liver, or soft tissue. In another aspect, the lesion is noncancerous.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, reducing or decreasing the size of a tumor can include reducing size as measured by volume, weight, cell number, decrease in number of living cells, increase in necrosis, or any other method for measuring size, or slowing or halting of, or reduction in growth. In an embodiment, volume of tumor size is calculated as length×width×width×0.52. In an embodiment, tumor size is decreased by between about 1% and about 100%. In an embodiment, tumor size is decreased by between about 25% and about 50%. In an embodiment, tumor size is decreased by about 50%, about 55%, about 60%, about 70%, about 75%, about 80%, about 85%, about 90%, about 95%, about 99% or about 100%. In an embodiment, tumor size is decreased by 50%, 55%, 60%, 70%, 75%, 80%, 85%, 90%, 95%, 99%, or 100%.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament” are used interchangeably herein, as context dictates, to refer to a compound or compounds or composition of matter which, when administered to a subject (human or animal) induces a desired pharmacological and/or physiologic effect by local and/or systemic action.

The terms “subject,” “individual,” and “patient” are used interchangeably herein, and refer to an animal, for example a human, to whom treatment with a pharmaceutical composition in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The formulations described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian. In another embodiment, the subject is any organism susceptible to cancer or tumors.

Conditions and disorders in a subject for which a particular drug or compound or composition (or combination thereof) is said herein to be “indicated” are not restricted to conditions and disorders for which that drug or compound or composition has been expressly approved by a regulatory authority, but also include other conditions and disorders known or reasonably believed by a physician to be amenable to treatment with that drug or compound or composition or combination thereof.

Compositions and methods described herein contemplate treating tumors, including reducing their size. “Tumor” has the meaning ordinarily understood in the art, and includes an abnormal mass of tissue, typically resulting when cells divide more than they should or do not die when they should. “Tumors” as referred to herein may be benign (not cancerous), or malignant (cancerous) and may occur any place in the body. “Lesion” as used herein refers to any abnormal area of tissue and can include, without limitation, benign and malignant tumors.

### Sclerosing Agents

In embodiments, the compositions, formulations, and methods for reducing tumor size described herein comprise sclerosing agents. Sclerosing agents act frequently by irritation of the venous intimal, or innermost, epithelium and are typically used in the treatment of varicose veins. More specifically, sclerosing agents cause a marked irritation or thrombosis with subsequent local inflammation and tissue necrosis and tissue contraction. Typically, the sclerosing agent, or sclerosant, is injected into a vein to entirely obliterate it. The agent damages the innermost lining of the vessel (the endothelium), resulting in a clot that blocks the blood circulation in the vein beyond, or creating scar tissue. In embodiments, the sclerosing agent comprises nonaethylene glycol monododecyl ether.

Nonaethylene glycol monododecyl ether is readily available and also can be referred to as C₁₂E₉, Dodecyl nonaethylene glycol ether, Dodecylnonaglycol, Polidocanol, Polyoxyethylene (9) lauryl ether, or HO(CH₂CH₂O)₉(CH₂)₁₁CH₃.

Its chemical structure can be depicted as:

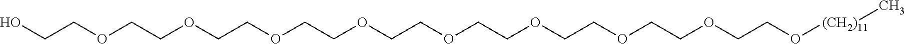

In embodiments, the nonaethylene glycol monododecyl ether is 98% nonaethylene glycol monododecyl ether. In embodiments, compositions in accordance with the present invention comprise about 1% of 98% nonaethylene glycol monododecyl ether by volume. In other embodiments, compositions in accordance with the present invention comprise 1% of 98% nonaethylene glycol monododecyl ether by volume. Other sclerosing agents known in the art can also be employed in the compositions and methods described herein, including, without limitation, Laureth-9 (polidocanol), ethanolamine oleate, morrhuate sodium, sodium tetradecyl sulfate, sterile talc powder, other suitable detergents, osmotic agents such as hypertonic sodium chloride solution and sodium chloride solution with dextrose, or chemical irritants, such as chromated glycerin and polyiodinated iodine, sclerosant foam formulations, alcohol, or other suitable sclerosing agents or derivatives, and combinations thereof.

### Penetrating Agents

In embodiments, the compositions, formulations, and methods described herein comprise a penetrating agent. Penetrating agents, also referred to herein as permeation enhancers, penetration enhancers, sorption promoters or accelerants, penetrate into skin to reversibly decrease the barrier resistance.

In embodiments, the penetrating agent comprises 1-methyl-2-pyrrolidinone. In embodiments, the 1-methyl-2-pyrrolidinone is anhydrous 1-methyl-2-pyrrolidinone. In embodiments, the 1-methyl-2-pyrrolidinone is 99.5% anhydrous 1-methyl-2-pyrrolidinone. 1-methyl-2-pyrrolidinone is readily available and is also referred to as 1-Methyl-2-pyrrolidone or N-Methyl-2-pyrrolidone. Its Empirical Formula (Hill Notation) is C₅H₉NO and its chemical structure can be depicted as:

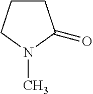

In embodiments, compositions and methods in accordance with the present invention comprise about 0.1% of 99.5% anhydrous 1-methyl-2-pyrrolidinone. In embodiments, compositions and methods in accordance with the present invention comprise 0.1% of 99.5% anhydrous 1-methyl-2-pyrrolidinone.

Numerous compounds have been evaluated in the art for penetration enhancing activity. Such compounds, suitable as penetrating agents in accordance with the compositions, formulations, and methods described herein include, without limitation, solvents, sulfoxides, Azones (e.g. laurocapram), other pyrrolidones (for example 2-pyrrolidone, 2P), alcohols and alkanols (e.g. ethanol, or decanol), glycols (for example propylene glycol, PG, a common excipient in topically applied dosage forms), surfactants (also common in dosage forms) and terpenes. Specifically, examples of the above and other penetrating agents suitable for use in accordance with the compositions and methods described herein include, without limitation, water, hydrocarbons, such as alkanes, alkenes, halogenated alkanes, squalene, squalene, and mineral oil; alcohols, such as alkanols, alkenols, glycols, polyglycols, and glycerols; acids, such as fatty acids, amines, cyclic and acyclic amides, such as azone and pyrrolidones, esters, such as isopropyl myristate, surfactants, including anionic, cationic, zwitterionic and non-ionic surfactants; terpenes, terpenoids, and essential oils; sulfoxides, such as dimethyl sulfoxide (DMSO) and its derivatives; lipids, such as phospholipids, and various other chemical groups, such as cyclic oligosaccharides such as cyclodextrins, amino acids and thioacyl derivatives of amino acids, alkyl amino esters and oxazolidinones, enzymes, such as papain and medicinal leech enzymes, ketones, others known in the art. Also contemplated within the scope of the present invention are tumor penetrating peptides, cell-penetrating peptides, and other suitable compounds and agents capable of penetrating skin, tissue and/or cell membranes. (See Karande, P. et al., (Review 2009), “Enhancement of transdermal drug delivery via synergistic action of chemicals”, Biochimica et Biophysica Acta, 1788: 2362-2373); Sugahara, K. N., et al., (May 21, 2010), “Co-administration of a tumor-penetrating peptide enhances the efficacy of cancer drugs”, Science, 328(5981): 1031-1035.)

### Acids

In embodiments, the compositions, formulations, and methods described herein comprise an acid (or salt thereof) and/or include methods wherein target tissue, such as that comprising a tumor contacted with a composition as described herein, comprises an acid (or salt thereof). As used herein, the term “acid” has a meaning in accordance with any of those commonly understood by one of ordinary skill in the art. An acid therefore includes, without limitation, a chemical substance whose aqueous solutions are characterized by a sour taste, the ability to turn blue litmus red, or the ability to react with bases and certain metals (like calcium) to form salts. Accordingly, acid salts, including pharmaceutically acceptable salts, are also contemplated and within the scope of the invention. Aqueous solutions of acids have a pH of less than 7.

Furthermore, acids suitable for use in accordance with the present invention include acids falling under the Lewis definition of an acid, i.e., a substance that can accept a pair of electrons to form a covalent bond, the Arrhenius Definition of an acid, i.e., substances which increase the concentration of hydrogen ions (H+), or hydronium ions (H₃O+), when dissolved in water, and the expansive Bronsted-Lowry Definition of an acid, i.e., a substance that can act a proton donor, and thus, any compound that can easily be deprotonated can be considered an acid.

An acid suitable for use in the invention and/or found naturally in tissue, therefore includes, without limitation, hydrochloric acid, lactic acid, a bile acid (including e.g., deoxycholic acid (deoxycholate), cholic acid, glycocholic acid, taurocholic acid, chenodeoxycholic acid, glycochenodeoxycholic acid, taurochenodeoxycholic acid, lithocholic acid), nucleic acids, fatty acids and fatty acid derivatives, amino acids, gastric acid, hyaluronic acid, ascorbic acid (Vitamin C), Bronsted and Lewis acids, carboxylic acids, halogenated carboxylic acids, acetylsalicylic acids, salicylic acids and variants and derivatives and salts thereof.

### Bile Acids

Bile acids, known in the art and suitable for use in the compositions and methods described herein and/or present in tissue comprising a tumor contacted by compositions and in methods in accordance with those described herein, include steroid acids found predominantly in the bile of mammals and other vertebrates.

Different molecular forms of bile acids can be synthesized in the liver by different species. Bile acids are conjugated with taurine or glycine in the liver, and the sodium and potassium salts of these conjugated bile acids are referred to as bile salts. (Russell D W (2003). “The enzymes, regulation, and genetics of bile acid synthesis”. Annu. Rev. Biochem. 72: 137-74; Chiang J Y (October 2009). “Bile acids: regulation of synthesis”. J. Lipid Res. 50 (10): 1955-66. Carey, M C.; Small, D M. (October 1972). “Micelle formation by bile salts. Physical-chemical and thermodynamic considerations”. Arch Intern Med. 130 (4): 506-27.)

Primary bile acids are those synthesized by the liver. Secondary bile acids result from bacterial actions in the colon. In humans, taurocholic acid and glycocholic acid (derivatives of cholic acid) and taurochenodeoxycholic acid and glycochenodeoxycholic acid (derivatives of chenodeoxycholic acid) are the major bile salts in bile and are roughly equal in concentration. The conjugated salts of their 7-alpha-dehydroxylated derivatives, deoxycholic acid and lithocholic acid, are also found, with derivatives of cholic, chenodeoxycholic and deoxycholic acids accounting for over 90% of human biliary bile acids. (See, e.g., Hofmann A F (1999). “The continuing importance of bile acids in liver and intestinal disease”. Arch. Intern. Med. 159 (22): 2647-58.)

Bile acids are about 80% of the organic compounds in bile (others are phospholipids and cholesterol). An increased secretion of bile acids produces an increase in bile flow. The main function of bile acids is to allow digestion of dietary fats and oils by acting as a surfactant that emulsifies them into micelles, allowing them to be colloidally suspended in the chyme before further processing. (See Hofmann A F, Borgström B (February 1964). “The intraluminal phase of fat digestion in man: the lipid content of the micellar and oil phases of intestinal content obtained during fat digestion and absorption”. J. Clin. Invest. 43: 247-57.) They also have hormonal actions throughout the body, particularly through the farnesoid X receptor and GPBAR1 (also known as TGR5) (See, e.g., Fiorucci S, Mencarelli A, Palladino G, Cipriani S (November 2009). “Bile-acid-activated receptors: targeting TGR5 and farnesoid-X-receptor in lipid and glucose disorders”. Trends Pharmacol. Sci. 30 (11): 570-80.)

### Bile Acids and Colon Cancer

Bile acids may have some importance in the development of colorectal cancer. (See, e.g., Degirolamo C, Modica S, Palasciano G, Moschetta A (2011). “Bile acids and colon cancer: Solving the puzzle with nuclear receptors”. Trends Mol Med. 17 (10): 564-72.) Deoxycholic acid (DCA) is increased in the colonic contents of humans in response to a high fat diet. (Reddy B S, Hanson D, Mangat S, et al. (September 1980). “Effect of high-fat, high-beef diet and of mode of cooking of beef in the diet on fecal bacterial enzymes and fecal bile acids and neutral sterols”. J. Nutr. 110 (9): 1880-7.) Experimental studies also suggest mechanisms for bile acids in colon cancer. Exposure of colonic cells to high DCA concentrations increase formation of reactive oxygen species, causing oxidative stress, and also increase DNA damage. (Bernstein H, Bernstein C, Payne C M, Dvorak K (July 2009). “Bile acids as endogenous etiologic agents in gastrointestinal cancer”. World J. Gastroenterol. 15 (27): 3329-40.)

In embodiments, the compositions, formulations, and methods of the present invention comprise an acid. In an embodiment, the acid comprises a bile acid. In another embodiment, the bile acid comprises deoxycholate or a salt thereof. In an embodiment, the composition comprises about 3% Na deoxycholate by weight. In an embodiment, the composition comprises 3% Na deoxycholate by weight. In an embodiment, the Na is dissolved in an alcohol. In an embodiment, the alcohol comprises benzyl alcohol. In an embodiment, the benzyl alcohol comprises about 3% benzyl alcohol, by volume. In an embodiment, the benzyl alcohol comprises 3% benzyl alcohol by volume. As described above, any other suitable acid, including other suitable bile acids, can be employed in accordance with the present invention.

### Alcohols

In embodiments, the compositions, formulations, and methods described herein comprise an alcohol. Alcohols include any organic compound in which the hydroxyl functional group (—OH) is bound to a carbon atom of an alkyl group.

In an embodiment, in the compositions, formulations, and methods described herein, the alcohol comprises benzyl alcohol. In an embodiment, the benzyl alcohol comprises about 3% benzyl alcohol by volume. In an embodiment, the benzyl alcohol comprises 3% benzyl alcohol, by volume.

Any other suitable alcohol can also be employed, including, without limitation any primary, secondary, or tertiary alcohol, any alkanol, alkenol, or any aromatic alcohol, any linear of branched alcohols of any size or chain length, any saturated or unsaturated alcohol, or any other compound that falls within the broad range of alcohols as understood by one of ordinary skill in the art. Examples of alcohols in addition to benzyl alcohol, suitable for use in the compositions, formulations, and methods described herein include, without limitation, linear alcohols such as methanol, ethanol, propanol, butanol, pentanol, hexanol, octanol, nonanol, decanol, or any other of any chain length that is known and readily available, other aromatic alcohols such as, without limitation, tryptophol, tyrosyl, and phenylethanol, or any other suitable alcohol.

### Pain Reducing Agents

In embodiments, the compositions, formulations, and methods described herein can comprise the inclusion or use of a pain-reducing agent in an amount effective to reduce pain in the subject. In an embodiment, the pain reducing agent comprises a general anesthetic. In an embodiment, the pain reducing agent comprises a local anesthetic. In another embodiment the pain reducing agent comprises lidocaine. In an embodiment, the composition comprises about 1% of 2% lidocaine. In another embodiment, the composition comprises 1% of 2% lidocaine. In embodiments, the pain reducing agent can be present in any preparation suitable for use in accordance with the compositions and methods described herein, including, without limitation, a 0.5%, 1%, 1.5%, 2%, 4%, or 5% injectable solution; or a 200, 400, or 800 mg/mL preparation. In an embodiment, the composition comprises between about 0.1% and about 1% of a pain reducing agent. In an embodiment, the composition comprises between about 1% and about 10% of a pain reducing agent.

In an embodiment, the pain reducing agent is comprised within the tumor reducing composition. In an embodiment, the pain reducing agent is administered separately from the tumor reducing composition.

Other suitable pain reducing agents for use in accordance with the present invention include, without limitation, procaine, bupivacaine, mepivacine, chloroprocine, tetracaine, ropivacaine, benzocaine, or any other suitable pain reducing agent known to one of ordinary skill in the art.

### Pharmaceutical Compositions

Described herein are pharmaceutical compositions comprising compounds of the invention and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or more therapeutic agents.

Thus, as used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Moreover, “Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopeia or other generally recognized pharmacopeia for use in animals and, more particularly, in humans.

In an embodiment, pharmaceutical compositions containing the therapeutic agent or agents described herein, can be, in one embodiment, administered to a subject by any method known to a person skilled in the art, such as, without limitation, parenterally, transmucosally, subcutaneously, intramuscularly, intravenously, intraarterially, intra-peritonealy, intra-cranially, intra-vaginally, or intra-tumorally. In one embodiment, the therapeutic agent or combination of therapeutic agents is administered intra-tumorally.

Carriers may be any of those conventionally used, as described above, and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. In one embodiment, water, preferably bacteriostatic water, is the carrier when the pharmaceutical composition is administered intravenously or intratumorally. Saline solutions and aqueous dextrose and glycerol solutions can also be employed as liquid carriers, particularly for injectable solutions.

Pharmaceutical compositions suitable for injectable use, including intratumoral injection, may include sterile aqueous solutions (where water soluble) or dispersions and sterile powders for the extemporaneous preparation of sterile injectable solutions or dispersion. For intravenous administration, suitable carriers include, without limitation, physiological saline, bacteriostatic water, Cremophor EL™ (BASF, Parsippany, N.J.) or phosphate buffered saline (PBS). The composition should be sterile and should be fluid to the extent that easy syringeability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as appropriate, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine Common salt-forming cations include, without limitation, ammonium, calcium, iron, magnesium, potassium, pyridinium, quaternary ammonium, sodium, and copper. Common salt-forming anions include, without limitation, acetate, carbonate, chloride, citrate, cyanide, fluoride, nitrate, nitrite, oxide, phosphate, and sulfate.

Compounds described herein also can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The compositions and formulations as described herein may be administered alone or with other biologically-active agents. Administration can be systemic or local, e.g. through portal vein delivery to the liver.

In one embodiment, the compositions are formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

It may be desirable to administer a pharmaceutical composition of the invention locally to the area in need of treatment; this may be achieved by, for example, and not by way of limitation, local infusion during surgery, by injection, by means of a catheter, by means of a suppository, or by means of an implant, said implant being of a porous, non-porous, or gelatinous material. According to some embodiments, administration can be by direct injection e.g., via a syringe, at the site of a tumor or neoplastic or pre-neoplastic tissue.

A compound of the present invention can be delivered in an immediate release or in a controlled release system. In one embodiment, an infusion pump may be used to administer a compound of the invention, such as one that is used for delivering chemotherapy to specific organs or tumors (see Buchwald et al., 1980, Surgery 88: 507; Saudek et al., 1989, N. Engl. J. Med. 321: 574). In another embodiment, a compound of the invention is administered in combination with a biodegradable, biocompatible polymeric implant, which releases the compound over a controlled period of time at a selected site. Examples of polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.). In yet another embodiment, a controlled release system can be placed in proximity of the therapeutic target, thus requiring only a fraction of the systemic dose.

Furthermore, at times, the pharmaceutical compositions formulated for parenteral administration may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Oils such as petroleum, animal, vegetable, or synthetic oils and soaps such as fatty alkali metal, ammonium, and triethanolamine salts, and suitable detergents may also be used for parenteral administration. The above formulations may also be used for direct intratumoral injection. Further, in order to minimize or eliminate irritation at the site of injection, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Moreover, to minimize or eliminate pain or discomfort resulting from injection or other administration, a pain reducing agent, as described herein, also may be administered either within the formulation, or separate from it.

The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described and known in the art.

### Effective Doses

Effective doses of the compositions of the present invention, for treatment of conditions or diseases vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, including cancer, also will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. In one embodiment, the dosage will be within the range of 0.01-1000 mg/kg of body weight. In another embodiment, the dosage will be within the range of 0.1 mg/kg to 100 mg/kg. In another embodiment, the dosage will be within the range of 1 mg/kg to 10 mg/kg. Effective doses may be extrapolated from dose-response curves derived from in vitro or animal model test bioassays or systems.

Moreover, suitable doses may also be influenced by permissible daily exposure limits (PDE) of any compound included in a formulation or method as described herein. Such limits are readily available, including, for example, from industry guidance recommendations provided periodically from the U.S. Food and Drug Administration, and the evaluation of these limits are within the knowledge and understanding of one of ordinary skill in the art.

As used herein, the term “administering” refers to bringing in contact with a compound of the present invention. Administration can be accomplished to cells or tissue cultures, or to living organisms, for example humans. In one embodiment, the present invention encompasses administering the compounds and compositions of the present invention to a human subject.

In one embodiment, methods of the present invention comprise the step of contacting one or more cells of said subject with a compound or a composition as described herein. In one embodiment, contacting one or more cells of a subject with a compound described herein comprises the step of administering a composition comprising said compound to said subject.

In one embodiment, the administration of a sclerosing agent and a penetrating agent, in accordance with the invention as described herein, may occur either simultaneously or time-staggered, either at the same site of administration or at different sites of administration.

In some embodiments, a timepoint comprises a point in time. In another embodiment, a timepoint comprises a time period shorter than 1 minute. In another embodiment, a timepoint comprises a time period shorter than 5 minutes. In another embodiment, a timepoint comprises a time period shorter than 30 minutes. A skilled artisan would appreciate that the terms “timepoint”, “time point” and “time period” may be used interchangeably having all the same qualities and meanings.

In an embodiment, any of the therapeutic or prophylactic drugs or compounds described herein may be administered simultaneously. In another embodiment, they may be administered at different timepoint than one another. In one embodiment, they may be administered within a few minutes of one another. In another embodiment, they may be administered within a few hours of one another. In another embodiment, they may be administered within 1 hour of one another. In another embodiment, they may be administered within 2 hours of one another. In another embodiment, they may be administered within 5 hours of one another. In another embodiment, they may be administered within 12 of one another. In another embodiment, they may be administered within 24 hours of one another.

In one embodiment, any of the therapeutic or prophylactic drugs or compounds described herein may be administered at the same site of administration. In another embodiment, they may be administered at different sites of administration.

The composition of the invention may be administered only once, or it may be administered multiple times. For multiple dosages, the composition may be, for example, administered three times a day, twice a day, once a day, once every two days, twice a week, weekly, once every two weeks, or monthly. Suitable dosage ranges and schedules can vary.

In an embodiment, a tumor in a subject is intratumorally injected on about day 1 and about day 3, on about day 8 and about day 10, on about day 15 and about day 17, on about day 22 and about day 24, on about day 29 and about day 31, and on about day 36. In an embodiment, dosing volume comprises about 50 ul of the composition per tumor during week one. In an embodiment, dosing volume comprises about 100 ul of the composition per tumor for week two. In an embodiment, dosing volume comprises about 200 ul of the composition for the remainder of the dosing administrations. In embodiments, dosing volume of the composition can range from about 5 ul to about 2000 ul of the composition per tumor. In embodiments, dosing volumes can range from about 5 ul to about 500 ul of the composition per tumor. In embodiments, dosing volume of the composition can range from about 10 ul to about 1000 ul of the composition per tumor. In embodiments, dosing volume of the composition can range from about 20 ul to about 2000 ul of the composition per tumor. In an embodiment, dosing volume comprises 50 ul of the composition per tumor during week one, 100 ul of the composition per tumor for week two, and 200 ul of the composition for the remainder of the dosing administrations. In an embodiment, dosing volume comprises 50 ul of the composition administered on day 1 and day 3, 100 ul of the composition per tumor administered on day 8 and day 10, and 200 ul of the composition administered on day 15, day 17, day 22, day 24, day 29, day 31, and day 36. (See Example 1). Determining other suitable dosing schedules and composition dosage ranges and amounts are within the skill of the ordinary artisan.

In embodiments, the tumor reducing compostions described herein comprises about 3% alcohol by volume, about 3% of an acid by weight (dissolved in the alcohol), about 1% of sclerosing agent by volume, about 0.1% penetrating agent, and QS bacteriostatic water. In embodiments, the tumor reducing compostions described herein comprises about 3% benzyl alcohol by volume, about 3% Na deoxycholate by weight (dissolved in the alcohol), about 1% of 98% nonaethylene glycol monododecyl ether by volume, about 0.1% of 99.5% anhydrous 1-methyl-2-pyrrolidinone, and QS bacteriostatic water. In an embodiment, the tumor reducing composition described herein comprises 3% benzyl alcohol by volume, 3% Na deoxycholate by weight (dissolved in the alcohol), 1% of 98% nonaethylene glycol monododecyl ether by volume, 0.1% of 99.5% anhydrous 1-methyl-2-pyrrolidinone, and QS bacteriostatic water. In an embodiment, the alcohol can be present in an amount between about 0.3% and about 30% by volume. In an embodiment, the acid can be present in an amount between about 0.3% and about 30% by volume. In an embodiment, the sclerosing agent can be present in an amount between about 0.1% and about 10% by volume. In an embodiment, the penetrating agent can be present in an amount between about 0.01% and about 1%.

It is to be noted that dosage values and amounts and ratios of individual components of the compositions described herein also may vary with the type and severity of the condition to be alleviated and other factors. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions described and contemplated herein can be included in a container, pack, or dispenser together with instructions for administration.

### Tumors, Cancer, and Other Disorders

In embodiments, where the terms “reducing a size of a tumor”, “treating or inhibiting a malignant cell proliferative disease or disorder”, “treating or inhibiting a non-solid cancer”, “treating or inhibiting a tumor”, “treating a lesion” are used herein in the description and in the claims, such terms are intended to encompass, without limitation, tumor formation, primary tumors, tumor progression, or tumor metastasis. Furthermore, the compounds, compositions, and methods described herein contemplate treatment of lesions, tumors, or tumor cells located in any tissue in which such lesions or tumors can occur in a subject. In embodiments, the compositions and methods described herein are suitable for use in disaggregating or removing any tissue to which the composition is applied and for which such result is desired.

In one embodiment, the methods of the present invention comprise inhibiting proliferation of cancer or tumor cells. In one embodiment, the term “inhibiting proliferation” in relation to cancer cells, in the context of the present invention refers to a decrease in at least one of the following: number of cells (due to cell death which may be necrotic, apoptotic or any other type of cell death or combinations thereof) as compared to control; decrease in growth rates of cells, i.e. the total number of cells may increase but at a lower level or at a lower rate than the increase in control; decrease in the invasiveness of cells (as determined for example by soft agar assay) as compared to control even if their total number has not changed; progression from a less differentiated cell type to a more differentiated cell type; a deceleration in the neoplastic transformation; or alternatively the slowing of the progression of the cancer cells from one stage to the next.

The term “treatment of cancer” or “treatment of a tumor” or “reducing size of a tumor” in the context of the present invention also includes at least one of the following: a decrease in the rate of growth of the cancer or tumor (i.e. the cancer or tumor still grows but at a slower rate); cessation of growth of the cancerous growth, i.e., stasis of the tumor growth, and, in one embodiment, the tumor diminishes or is reduced in size. The term also includes reduction in the number of metastasis, reduction in the number of new metastasis formed, slowing of the progression of cancer from one stage to the other and a decrease in the angiogenesis induced by the cancer. In one embodiment, the tumor is totally eliminated. Additionally, included in this term is lengthening of the survival period of the subject undergoing treatment, lengthening the time of diseases progression, tumor regression, and the like. This term also encompasses prevention for prophylactic situations or for those individuals who are susceptible to contracting a tumor. In embodiments, the administration of the compounds of the present invention will reduce the likelihood of the individual contracting the disease. In one embodiment, the individual to whom the compound is administered does not contract the disease.

Thus, in one embodiment, treating may include directly affecting or curing, suppressing, inhibiting, preventing, reducing an incidence, reducing the severity of, delaying the onset of, reducing symptoms associated with the disease, disorder or condition, or a combination thereof. Thus, in one embodiment, “treating” refers, inter alia, to delaying progression, expediting remission, inducing remission, augmenting remission, speeding recovery, increasing efficacy of or decreasing resistance to alternative therapeutics, or a combination thereof. In another embodiment, treating refers to reducing the pathogenesis of, ameliorating the symptoms of, ameliorating the secondary symptoms of, or prolonging the latency to a relapse of a cancer in a subject. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, a subject as described herein has a pre-cancerous condition. In another embodiment, a subject as described herein has a benign hyperproliferative disorder. In another embodiment, a subject has cancer.

In one embodiment, the term “pre-cancer” or “pre-malignant” as used herein interchangeably refers to diseases, syndromes or other conditions associated with an increased risk of cancer. Pre-cancer conditions in the context of the present invention include, but are not limited to: breast calcifications, vaginal intra-epithelial neoplasia, Barrett's esophagus, atrophic gastritis, dyskeratosis congenital, sideropenic dysphagia, lichen planus, oral sibmucous fibrosis, actinic keratosis, solar elastosis, cervical desplasia, leukoplakia and erythroplakia, or other tumor as described herein.

In one embodiment, the term “benign hyperproliferative disorder” as used herein refers to a condition in which there is an abnormal growth and differentiation of cells and an increase in the amount of organic tissue that results from cell proliferation. The benign hyperproliferative disorder may be attributed to lack of response or inappropriate response to regulating factors, or alternatively to dysfunctional regulating factors. Non-limiting example of benign hyperproliferative disorder are psoriasis and benign prostatic hyperplasia (BPH).

In another embodiment, the subject has a lymphoma. In one embodiment, lymphomas develop in the glands or nodes of the lymphatic system, a network of vessels, nodes, and organs (in one embodiment, the spleen, tonsils, and thymus) that purify bodily fluids and produce lymphocytes, which, in one embodiment, comprise infection-fighting white blood cells. In one embodiment, lymphomas are “solid cancers.” In another embodiment, a lymphoma may occur in a specific organ such as the stomach, breast or brain. In one embodiment such a lymphoma is an extranodal lymphoma.

In one embodiment, a subject as described herein comprises a Mixed Type cancer. In one embodiment, a mixed type cancer comprises several types of cells. In one embodiment, the type components may be within one category or from different categories. In one embodiment a Mixed Type cancer comprises adenosquamous carcinoma, mixed mesodermal tumor, carcinosarcoma, teratocarcinoma, or a combination thereof.

As used herein, the term “cancer” includes the above categories of carcinoma, sarcoma, myeloma, leukemia, lymphoma and mixed type tumors. In particular, the term cancer includes: lymphoproliferative disorders, breast cancer, ovarian cancer, prostate cancer, cervical cancer, endometrial cancer, lung cancer, bone cancer, liver cancer, stomach cancer, bladder cancer, colon cancer, colorectal cancer, pancreatic cancer, cancer of the thyroid, head and neck cancer, cancer of the central nervous system, brain cancer, cancer of the peripheral nervous system, skin cancer, kidney cancer, as well as metastases of all the above. More particularly, as used herein the term may refer to: hepatocellular carcinoma, hematoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

In another embodiment, the cancer is an adenocarcinoma of the stomach or gastroesophageal junction, Dermatofibrosarcoma protuberans, Endocrine/neuroendocrine tumors, Gastrointestinal stromal tumor, Giant cell tumor of the bone, Kaposi sarcoma, Myelodysplastic/myeloproliferative disorders, Ovarian epithelial/fallopian tube/primary peritoneal cancers, Soft tissue sarcoma, Systemic mastocytosis, Germ cell tumor, or a combination thereof.

In one embodiment, the subject having cancer or a tumor has been treated with surgery, chemotherapy, radiation therapy, a targeted therapy, including therapies that are intended to boost immune system responses against cancer, or a combination thereof.

In one embodiment, the tumor is a solid tumor. In one embodiment, the solid tumor is a colon carcinoma, prostate cancer, breast cancer, lung cancer, skin cancer, liver cancer, bone cancer, ovary cancer, pancreas cancer, brain cancer, head and neck cancer or other solid tumor.

In one embodiment, the cancer, non-cancerous tumor, or other lesion is in a tissue of the breast, prostate, lung, colon, stomach, pancreas, ovary, brain, skin, bone, fat, lymph, gastrointestinal tract, liver, or soft tissue. In another embodiment, the tumor is in a tissue that comprises gastrointestinal (GI) tract tissue. In another aspect, the GI tract tissue comprises tissue of the anus, rectum, colon, esophagus, stomach, mouth, pharynx, small intestine, liver, pancreas, or biliary tract. In another embodiment, the cancer is a hematopoietic cancer, a neuroblastoma, or a malignant glioma.

In an embodiment, the tumor or lesion is, without limitation, an adenoma, a hemangioma, a cherry angioma, a lipoma, a lipoblastoma, a hibernoma, a fibroma, a meningioma, a myoma, a leiomeyoma, a rhabdomyoma, a nevi (mole), a neuroma, a neurofibroma, schwannoma, an osteochondroma, a papilloma, seborrheic keratoses, acrochordon, sebaceous hyperplasia, a dermatofibroma, an angiolipoma, a cyst, an epidermal inclusion cyst, a milium, a soft tissue lesion, or a granuloma.

### Combined Treatments

In another embodiment, any of the methods of the present invention may further comprise the step of contacting one or more cells of said subject with an anti-cancer treatment, or otherwise include a combination treatment or therapy. In one embodiment, the anti-cancer treatment is radiotherapy.

In another embodiment, the anti-cancer treatment is an anti-cancer drug. In one embodiment, the anti-cancer drug is a chemotherapeutic.

In one embodiment, the chemotherapeutic comprises 5-fluorouracil, Bleomycin, capecitabine, cisplatin, Cyclophosphamide, dacarbazine, Doxorubicin, Epirubicin, etoposide, folinic acid, Methotrexate, Mustine, oxaliplatin, prednisolone, procarbazine, vinblastine, vincristine, or a combination thereof.

In another embodiment, the present invention provides a method of treating, inhibiting, or suppressing a cancer or tumor in a subject comprising contacting one or more cells of said subject with a tumor reducing composition as described herein and a targeted therapy.

### Targeted Therapies

In one embodiment, the present invention provides methods of treating cancer comprising administering a composition as described herein in combination with one or more targeted therapies.

In one embodiment, an immunotherapeutic compound is targeted to particular molecules expressed abnormally by cancer cells. In one embodiment, the targeted therapy comprises a hormone therapy, signal transduction inhibitor, gene expression modulator, apoptosis inducer, angiogenesis inhibitor, immunotherapy, or toxin delivery molecules.

In one embodiment, the targeted therapy utilizes small molecules. In another embodiment, the targeted therapy utilizes antibodies, which, in one embodiment, are monoclonal antibodies.

In one embodiment, the immunotherapeutic compound comprises abiraterone acetate (Zytiga®), ado-trastuzumab emtansine (Kadcyla®), afatinib dimaleate (Gilotrif®), alectinib (Alecensa®), alemtuzumab (Campath®), Alitretinoin (Panretin®), anastrozole (Arimidex®), Atezolizumab (Tecentriq™), axitinib (Inlyta®), belinostat (Beleodaq®), Bevacizumab (Avastin®), bexarotene (Targretin®), blinatumomab (Blincyto®), bortezomib (Velcade®), bosutinib (Bosulif®), brentuximab vedotin (Adcetris®), Cabazitaxel (Jevtana®), cabozantinib (Cabometyx™), Cabozantinib (Cometriq®), carfilzomib (Kyprolis®), ceritinib (LDK378/Zykadia™), Cetuximab (Erbitux®), cobimetinib (Cotellic™), crizotinib (Xalkori®), dabrafenib (Tafinlar®), daratumumab (Darzalex™), dasatinib (Sprycel®), denileukin diftitox (Ontak®), Denosumab (Xgeva®), Dinutuximab (Unituxin™), elotuzumab (Empliciti™) enzalutamide (Xtandi®), Erlotinib (Tarceva®), everolimus (Afinitor®), exemestane (Aromasin®), fulvestrant (Faslodex®), gefitinib (Iressa®), Ibritumomab tiuxetan (Zevalin®), ibrutinib (Imbruvica®), idelalisib (Zydelig®), Imatinib mesylate (Gleevec®), Ipilimumab (Yervoy®), ixazomib citrate (Ninlaro®), Lanreotide acetate (Somatuline® Depot), lapatinib (Tykerb®), lenvatinib mesylate (Lenvima®), letrozole (Ferrara®), necitumumab (Portrazza™), nilotinib (Tasigna®), nivolumab (Opdivo®), obinutuzumab (Gazyva®), ofatumumab (Arzerra®), olaparib (Lynparza™), olaratumab (Lartruvo™), osimertinib (Tagrisso™), palbociclib (Ibrance®), panitumumab (Vectibix®), panobinostat (Farydak®), pazopanib (Votrient®), pembrolizumab (Keytruda®), pertuzumab (Perjeta®), pralatrexate (Folotyn®), radium 223 dichloride (Xofigo®), ramucirumab (Cyramza®), regorafenib (Stivarga®), rituximab (Rituxan®), romidepsin (Istodax®), ruxolitinib phosphate (Jakafi®), siltuximab (Sylvant®), sonidegib (Odomzo®), sorafenib (Nexavar®), sunitinib (Sutent®), tamoxifen (Nolvadex), temsirolimus (Torisel®), toremifene (Fareston®), trametinib (Mekinist®), Trastuzumab (Herceptin®), Tretinoin (Vesanoid®), vandetanib (Caprelsa®), vemurafenib (Zelboraf®), venetoclax (Venclexta™), Vismodegib (Erivedge®), vorinostat (Zolinza®), ziv-aflibercept (Zaltrap®), or a combination thereof.

In another embodiment, methods of the present invention further comprise the step of contacting one or more cells of the subject with an immunotherapeutic compound.

### Immunotherapeutic Compounds

In an embodiment, an immunotherapy as described herein is a monoclonal antibody that recognizes specific molecules on the surface of cancer cells. In one embodiment, binding of the monoclonal antibody to the target molecule results in the immune destruction of cells that express that target molecule. In another embodiment, the antibody binds to certain immune cells to enhance their actions on cancer cells.

In one embodiment, the immunotherapeutic compound comprises imatinib or trastuzumab. In one embodiment, the immunotherapeutic compound comprises a checkpoint inhibitor. In one embodiment, the checkpoint inhibitor comprises a Programmed cell Death protein 1 (PD1) inhibitor or a Programmed cell Death Ligand 1 (PD-L1) inhibitor. In one embodiment, the PD-1 or PD-L1 inhibitor is an antibody. In one embodiment, the antibody comprises Nivolumab, Pembrolizumab, Pidilizumab, Avelumab, BMS 936559, or MPDL328OA. In one embodiment, the immunotherapeutic compound comprises chimeric antigen receptor T cells (CAR T-cells).

Each of the above additives, excipients, formulations and methods of administration represents a separate embodiment of the present invention.

All patents, patent applications, and scientific publications cited herein are hereby incorporated by reference in their entireties.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

In a study of 12 mice in which Ct26 tumors were implanted, 6 received a control injection of bacteriostatic water and 6 received the experimental formulation set forth in Example 1.

### Example 1

The experimental formulation was comprised of:
1. 3% benzyl alcohol by volume
2. 3% Na deoxycholate by weight (dissolved in the alcohol)
3. 1% of 98% nonaethylene glycol monododecyl ether by volume
4. 0.1% of a 99.5% anhydrous 1-methyl-2-pyrrolidinone
5. QS with bacteriostatic water

### Example 2

### Methods

1. After an acclimation period of 3-5 days, 15 BalB/C female mice were inoculated in the right flank with 1 million of CT26 cells (suspended in 100 ul 1×BH₂O).
2. Beginning at Day 4 post cell inoculation, the tumor volume was measured every day until their average volume reached 100 mm3 (Volume=length×width×width×0.52).
3. Twelve (12) tumor bearing mice with preferred tumor volume were selected and randomly grouped into 2 groups (n=6 per group) and individually identified (tail mark or ear tag).
4. Mice were weighed and intratumorally injected with 1×BH₂O or the experimental formulation on Day 1 and 3, then on Day 8 and 10, Day 15 and 17, Day 22 and 24, Day 29 and 31, and Day 36.
5. The dosing volume of 1×BH₂O or experimental formulation was at 50 ul each tumor for first week, 100 ul for each tumor for 2nd week, 200 ul per tumor for the rest of dosing.
6. Tumor volume and body weight of mice were measured twice a week until the termination of the study or the group average tumor volume of the control group reached 1500-2000 mm3 at which the study was be terminated

At the end of a 3-week observation period post dosing, the control animals had tumor growth greater than 2000 m³, while two experimental animals had no tumors and 4 had dramatically smaller tumor volumes than controls. All animals were sacrificed and histopathology of tumor sites was assessed. Data is presented in Example 3

### Example 3

### Histopathologic Comparison of Control Versus Treated Xenografts in Mice

### Introduction

The purpose of the study set forth in this Example was to assess the histopathology of tumors from mice treated with the formulation set forth in Example 1 by the methods set forth in Example 2

### Materials and Methods

Xenograft tumors (N=11) were presented for histopathologic examination. The tissues were prepared using standard CBI methodology. Tumors were gross trimmed and processed, then embedded in paraffin. Blocks were microtomed at 5 μm and were stained with hematoxylin-eosin. Tissues were examined histopathologically by a board certified veterinary pathologist. All tissues were in good condition. Minimal to no artifactual changes as a result of tissue handling were present.

### Results

The morphology of the tumors was consistent with the xenograft cell line. There were differences between the Control (comprising bacteriostatic water) and the tumors treated with the experimental formulation in that the treated tumors were clearly smaller and had a much larger area of necrosis than that of the control tumors. The individual findings are presented in Table 1 below.

It should be noted that two of the treated animals lost all appearance of tumor and their tissues were not included in the histology study.

TABLE 1
Histopathologic findings.

| | Animal | | Intratumoral |
|---|---|---|---|
| Group | ID | Tumor Size | necrosis |
| Control | G1A-1 | large | moderate |
| | G1A-2 | large | mild |
| | G1B-2 | large | mild |
| | G1B-3 | very large | moderate |
| | G1B-4 | large | moderate |
| Treated | G2A-1 | small | severe |
| | G2A-3 | small | severe |
| | G2B-1 | small | severe |
| | G2B-3 | small | severe |
| | G2B-5 | moderate | severe |

Having described preferred embodiments of the invention, it is to be understood that the invention is not limited to the precise embodiments, and that various changes and modifications may be effected therein by those skilled in the art without departing from the scope or spirit of the invention as defined in the appended claims.

1. A pharmaceutical composition comprising a combination of a sclerosing agent and a penetrating agent.

2. The pharmaceutical composition of claim 1, wherein the sclerosing agent is nonaethylene glycol monododecyl ether.

3. The pharmaceutical composition of claim 1, wherein the penetrating agent is anhydrous 1-methyl-2-pyrrolidinone.

4. The pharmaceutical composition of claim 1, wherein the sclerosing agent and the penetrating agent are present in combination in an amount effective to reduce a size of a tumor in a subject.

5. The composition of claim 4, wherein the subject is a mammal.

6. The composition of claim 5, wherein the mammal is a human.

7. The composition of claim 1, further comprising an alcohol.

8. The composition of claim 7, wherein the alcohol is benzyl alcohol.

9. The composition of claim 1, further comprising an acid or a salt thereof.

10. The composition of claim 9, wherein the acid is a bile acid.

11. The composition of claim 10, wherein, the bile acid salt is sodium deoxycholate.

12. The composition of claim 1, further comprising a pain reducing agent.

13. The composition of claim 12, wherein the pain reducing agent is lidocaine.

14. The composition of claim 1, wherein the combination penetrates a tissue in presence of an acid.

15. A pharmaceutical composition comprising a bile acid, nonaethylene glycol monododecyl ether, and anhydrous 1-methyl-2-pyrrolidinone.

16. The pharmaceutical composition of claim 15, wherein the bile acid is sodium deoxycholate.

17. A method of reducing a size of a tumor in a subject, comprising contacting a tumor with a composition comprising a therapeutically effective amount of a combination of a sclerosing agent and a penetrating agent.

18. A method of reducing a size of a tumor in a subject, comprising contacting a tumor with a composition comprising a therapeutically effective amount of a combination of a sclerosing agent and a penetrating agent, and a bile acid.

19. The method of claim 17, wherein the contacting comprises intratumoral injection.

20. The method of claim 17, wherein the tumor is present in a tissue of the breast, prostate, lung, colon, stomach, pancreas, ovary, brain, skin, bone, fat, lymph, gastrointestinal tract, liver, or soft tissue.

21. The method of claim 17, wherein the sclerosing agent is nonaethylene glycol monodecyl ether.

22. The method of claim 17, wherein the penetrating agent is anhydrous 1-methyl-2-pyrrolidinone.

23. The method of claim 18, wherein the bile acid is deoxycholic acid, cholic acid, glycocholic acid, taurocholic acid, chenodeoxycholic acid, glycochenodeoxycholic acid, taurochenodeoxycholic acid, or lithocholic acid.

24. The method of claim 17, wherein the subject is a mammal.

25. The method of claim 24, wherein the mammal is a human.

26. The method of claim 17, wherein the composition further comprises an alcohol.

27. The method of claim 26, wherein the alcohol is benzyl alcohol.

28. The method of claim 17, wherein the composition further comprises a pain reducing agent.

29. The method of claim 28, wherein the pain reducing agent is lidocaine.

30. The method of claim 18, wherein the composition comprises benzyl alcohol, sodium deoxycholate, nonaethylene glycol monododecyl ether, and anhydrous 1-methyl-2-pyrrolidinone.

31. The method of claim 17, wherein the tumor contacted with the composition comprises an increase in intratumoral necrosis compared to a tumor contacted with a control comprising bacteriostatic water.

32. The method of claim 17, wherein the tumor is cancerous.

33. A method of treating a lesion in a subject, comprising contacting the lesion with the pharmaceutical composition of claim 1.

34. The method of claim 33, wherein the lesion is present in a tissue of the breast, prostate, lung, colon, stomach, pancreas, ovary, brain, skin, bone, fat, lymph, gastrointestinal tract, liver, or soft tissue.

35. The method of claim 33, wherein the lesion is noncancerous.
